(19) United States
(12) Patent Application Publication
(10) Pub. No.: US 2005/0059682 A1
(43) Pub. Date: Mar. 17, 2005

(54) Compositions and methods for treatment of cancer

(75) Inventor: Joseph Rubinfeld, Danville, CA (US)

Correspondence Address: WILSON SONSINI GOODRICH & ROSATI, 650 PAGE MILL ROAD, PALO ALTO, CA, 943041050, US

(73) Assignee: SuperGen, Inc., a Delaware Corporation, Dublin, CA (US)

(21) Appl. No.: 10/661,386

(22) Filed: Sep. 12, 2003

(51) Int. Cl. A61K 31/52

(52) U.S. Cl. 514/263.1

(57) ABSTRACT

Compositions and methods for treatment of conditions related to the overexpression of EZH2, such as late stage prostate cancer, using a DNA methylation inhibitor and/or a histone deacetylase inhibitor, optionally in combination with an EZH2 antagonist and/or an antineoplastic agent, to specifically target diseases associated with EZH2 over-expression. Further provided are reagents and kits for treatment of EZH2 overexpression.

### BACKGROUND

This invention relates to compositions and methods of treating cancer. In particular, this invention relates to compositions and methods of treatment of prostate cancer, especially metastatic prostate cancer.

Prostate cancer is the second most common diagnosed cancer in men in the United States after lung cancer. Roughly 190,000 men are diagnosed with prostate cancer in the United States and nearly 30,000 men die from the disease yearly. Statistics are similar in Europe, where in England and Wales roughly 18,000 men are diagnosed with prostate cancer and approximately 8,500 men die from the disease each year.

The causes of prostate cancer are not well understood. However, there are certain risk factors that can elevate a man's susceptibility to prostate cancer. These factors include, for example, age, family history of prostate cancer, race, testosterone level, diet and dietary factors.

The greatest risk factor for prostate cancer is age. Prostate cancer tends to affect older men. For example, in the United States, prostate cancer is found mainly in men over the age of 55. The average age of patients at the time of diagnosis with prostate cancer is 75. By the age of 80, about half of all men will have some form of prostate cancer, though most will die from other causes.

A family history of prostate cancer is the second greatest risk factor for developing the prostate cancer. Prostate cancer often clusters in families and approximately 5-10% of the cases are estimated to have a substantial inherited component. It is also estimated that a strong predisposing gene could be responsible for roughly 43% of the cases by age 55. The relative risk of prostate cancer is increased two-fold with one first-degree relative (e.g., father or brother) diagnosed with prostate cancer at the age of 70 or under. The risk rises to four-fold with two relatives diagnosed with prostate cancer if one of them is diagnosed at the age of 65 or under. The risk with three or more relatives diagnosed with prostate cancer is increased seven-fold.

Race is also a risk factor for prostate cancer. It is estimated that the rate of incidence of prostate cancer is 62 percent higher in African American men than in Caucasian men. Also, the mortality rate is twice as high in African American men than in Caucasian men. Asian and American Indian men have the lowest incidences of prostate cancer.

Hormonal influences may also play a role in the development of prostate cancer. Estrogen and androgen deprivation can cause tumor regression. While, on the other hand, high levels of testosterone may attribute to prostate cancer.

Finally, there is some evidence suggesting that a diet high in animal fat may increase the risk of developing prostate cancer. On the other hand, a diet high in fruits and vegetables may decrease the risk.

Prostate cancer can behave differently in different men. For example, some prostate cancer can be present in small deposits within the prostate gland and may remain dormant for many years (e.g., benign tumors). While other prostate cancer can grow rapidly and spread to other parts of the body, in particular bones (e.g., metastatic cancer). While the latter form of prostate cancer may be extremely lethal and require quick intervention treatment, the former may be dormant for many years and require only occasional monitoring. This makes the diagnosis, prognosis and treatment of prostate cancer very difficult.

Early diagnosis of prostate cancer can be achieved by digital rectal examination, transrectal ultrasound and/or serum prostate-specific antigen (PSA). PSA is the primary method of testing for prostate cancer and is accomplished by evaluating the level of PSA in an individual's blood. However, the PSA test is not a perfect test. Approximately two-third of men who have raised levels of PSA do not have prostate cancer. The PSA test also cannot distinguish between men who have slow-growing prostate cancer and those who have a metastatic form of the disease. Furthermore, there are different PSA tests available today and numerous laboratories that process them. It is estimated that there is up to a 30% difference in results of PSA tests between different tests and between different laboratories.

Other symptoms of prostate cancer that may be useful in diagnosis and prognosis of the disease include, for example, difficulty in passing urine, a need to urinate more frequently, inability to urinate, weak or interrupted flow of urine, painful or burning urination, blood in the urine and/or frequent pain or stiffness in the lower back, hips or upper thighs.

Generally, treatment of early-stage prostate cancer involves active monitoring of a patient. This allows a urologist to decide if and when to offer more radical treatment such as surgery or radiotherapy. Surgery can include removal of all or part of the prostate. In radical prostatectomy a doctor removes the entire prostate gland to cure the disease. This treatment is not usually recommended for men with less than 10 years life expectancy as complications include operative mortality, impotence and incontinence as well as post-operative sexual dysfunction. Reported frequencies of post-prostatectomy impotence range from 20-80%; reported incidences of post-prostatectomy incontinence range from 4-21% for mild or stress incontinence and from 0-7% for total incontinence eighteen months post-operative.

Radiation-therapy uses high-energy X-rays to kill cancer cells and aims to cure the disease. But, like surgery, radiation is generally not recommended for men with less than 10 years life expectancy due to short-term and long-term complications. Short-term side effects from radiation therapy include bowel and bladder problems. Longer-term complications include impotence and urinary problems. Reports of impotence after radiation therapy range from 25-60% and reports of incontinence after radiation therapy range from 0-5%. Furthermore, approximately 10% of patients experience diarrhea or bowel problems requiring treatment and up to 30% have occasional episodes of rectal bleeding after radiation therapy.

Furthermore, hormonal therapy may be administered after surgery or radiation to keep cancer cells from growing or to keep cancer cells from coming back. Hormonal therapy can be achieved by orchiectomy (castration), administration of luteinizing hormone-releasing hormone agonists that prevent the testicles from producing testosterone (e.g., leuprodline, goserelin, and buserelin), administration of antiandrogen that block the action of androgens (e.g., flutamide and bicalutadmide) and administration of drugs that prevent the adrenal glands from making androgens (e.g., ketoconazole and aminoglutethimide).

Currently, there is no cure for locally advanced or metastatic prostate cancer. Frequently, orchiectomy and other hormone treatments (estrogen) are used to treat metastatic prostate cancer. Other drugs, such as diethylstilbestrol, ketoconazole and cyproterone acetate are also used in an attempt to blockade the adrenal source of testosterone and prevent growth of metastatic prostate cancer. An alternative treatment includes suramin sodium, a polysulfonated naphthylurea that binds the epidermal group factor receptor (EGFR) and blocks cellular growth. Suramin sodium has been shown to decrease circulating androstenedion, dihydroepiandrosterone and dihydroepiandrosterone sulfate by 40% in patients with metastatic prostate cancer after previous hormone therapy has failed. However, these treatments provide only limited success and many patients fail to respond to these treatments.

Single-agent chemotherapy has also produced little or not effect on the treatment of prostate cancer. Studies of doxorubicin, mitoxantrone, cisplatin, cyclophosphamide, methotrexate, estramustine and 5-fluorouracil have demonstrated minimal efficacy of such single agents. Similarly, combination treatment of estramustrine and paclitaxel has produced a 53% response rate but with greater than grade 2 toxicity in approximately one third of all patients.

As a result of the risks and side effects associated with prostate cancer treatments, it is debatable whether men should be treated for early stages of prostate cancer, especially if the cancer does not cause problems throughout their lifetime. Therefore, there exists a need for effective treatment of human prostate cancer that does not involve serious complications and/or side effects, in particular, late-stage, metastatic and hormone-refractory prostate cancer. The present invention relates to one such improved drug regimen for treating cancer, especially, prostate cancer, and in particular, late-stage metastatic prostate cancer.

### SUMMARY OF THE INVENTION

The present invention provides new and improved compositions and methods of treatment of disease associated with the over-expression of EZH2. In particular, the present invention provides new and improved compositions and methods of treatment of cancers associated with the over-expression of EZH2 such as prostate cancer and B-cell non-Hodgkin's lymphomas.

In one aspect of the invention, a method is provided for treating a patient who is suffering from a disease associated with over-expression of EZH2.

In one embodiment, the method comprises: administering to the patient a therapeutically effective amount of a DNA methylation inhibitor. Optionally, the method may further comprise administering to the patient a histone deacetylase inhibitor, an EZH2 antagonist, and/or an antineoplastic agent.

In another embodiment, the method comprises: administering to the patient a therapeutically effective amount of a histone deacetylase inhibitor. Optionally, the method may further comprise administering to the patient a DNA methylation inhibitor, an EZH2 antagonist, and/or an antineoplastic agent.

According to any of the above embodiments, the method may further comprise detecting a level of EZH2 expression in the patient. EZH2 expression levels may be detected in vivo by using various imaging methods or ex vivo in a sample derived from the patient, e.g., a biopsy taken from the patient's prostate. Quantitation of EZH2 expression levels may be achieved by measuring levels of EZH2 MRNA or EZH2 protein expressed in the cells of the sample. EZH2 expression levels may be detected prior to, during, or post administration of any of the above-described agents, i.e., the DNA methylation inhibitor, the histone deacetylase inhibitor, the EZH2 antagonist and the antineoplastic agent. Preferably, EZH2 expression levels are detected prior to the administration of an agent so as to ascertain severity or stage of the disease. Further, the EZH2 expression levels may be monitored throughout the course of the treatment to check the efficacy of the treatment and/or prognosis of the disease.

According to any of the above embodiments, the DNA methylation inhibitor is a cytidine analog or derivative thereof. Examples of cytidine analogs or derivatives include, for example, 5-azacytidine and 5-aza-2′-deoxycytidine (“decitabine”). In a preferred embodiment, the DNA methylation inhibitor is decitabine.

According to any of the above embodiments, the histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB))

According to any of the above embodiments, the EZH2 antagonists is an EZH2 antisense nucleic acid, a ribozyme against EZH2 nucleic acid, a triple helix against EZH2 nucleic acid, a siRNA against EZH2 an EZH2 antibody, an EZH2 binding polypeptide or a compound that specifically inhibits activities of EZH2 nucleic acid or protein.

According to any of the above embodiments, the anti-neoplastic agent may be an alkylating agent, an antibiotic agent, a retinoid, an antimetabolic agent, a hormonal agent, a plant-derived agent, or a biologic agent.

According to any of the above embodiments, the disease associated with over-expression of EZH2 may be a hematological disorder, a cancer, or any other disorder.

Hematologic disorders include abnormal growth of blood cells that can lead to dysplastic changes in blood cells and hematological malignancies such as various leukemias. Examples of hematological disorders include, but are not limited to, acute myeloid leukemia, acute promyelocytic leukemia, acute lymphoblastic leukemia, chronic myelogenous leukemia, the myelodysplastic syndromes, and sickle cell anemia.

Examples of cancers include, but are not limited to, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas. Examples of lymphomas include, for example, small lymphocytic lymphoma, follicular lymphoma, large B-cell lymphoma, mantle-cell lymphoma, and Burkitt lymphoma.

According to any of the above embodiments, the method can be used to treat a disease associated with EZH2 over-expression at any stage of the disease, early, middle, or late stage. In particular, the method may be used to treat prostate cancer, especially prostate cancer in its later stages, e.g., when it becomes hormone refractory or metastatic. The method may also be used to treat lymphoma, in particular, B-cell non-Hodgkin's lymphoma with manifestation of EZH2 over-expression.

In a preferred embodiment, the DNA methylation inhibitor or the EZH2 antagonist is administered prior to administering the histone deacetylase inhibitor.

The levels of EZH2 expression may further be used to determine the amount of EZH2 antagonists, DNA methylation inhibitors, histone deacetylase inhibitors, and anti-neoplastic agents to be administered. EZH2 expression levels can be determined by taking a biopsy. In cases of prostate cancer, for example, the biopsy may be taken directly from a patient's prostate. In cases of non-Hodgkin's lymphoma, the biopsy may be taken from a patient's lymph node. The level of EZH2 expression in the biopsy is then compared with a control level of expression. A control level of expression can be a level of expression in a tissue sample derived from another part of the patient's body, a tissue sample derived from a healthy individual, a previous sample taken from the patient, or known levels of EZH2 expression in a healthy individual. Expression levels can be determined using any known technique including Northern blots and Western blots. If the level of EZH2 expression in the biopsy is greater, by a statistically significant amount, from the level of EZH2 expression in a control, then the patient is treated with a therapeutically effective amount of any of the following: DNA methylation inhibitors, histone deacetylase inhibitors, antineoplastic agents, EZH2 antagonists, or a combination thereof. Combination treatment may require smaller dosages due to the synergetic effect of any of the above compositions. Generally, the greater the EZH2 expression in a patient, the greater the dosage or the longer the treatment course. The level of EZH2 expression in a patient can be determined prior to treatment, during treatment or post treatment. EZH2 expression may be useful in diagnosing a particular type of disease or stage of the disease, as well as to verify efficacy of treatment.

The DNA methylation inhibitors, the histone deacetylase inhibitors, the anti-neoplastic agents, and the EZH2 antagonists may be delivered via various routes of administration. For example, they may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms. In a preferred embodiment, the DNA methylation inhibitor is administered intravenously or subcutaneously, and the histone deacetylase inhibitor is administered intravenously.

In some preferred embodiments, the DNA methylation inhibitor is decitabine and is administered into a patient via an intravenous (“i.v.”) infusion for 1-24 hours per day, 3-5 days per treatment cycle, at a dose optionally ranging from 1-100 mg/m², optionally ranging from 2-50 mg/m², and optionally ranging from 5-20 mg/m². The preferred dosage below 50 mg/m² for decitabine is considered to be much lower than that used in conventional chemotherapy for cancer.

Optionally, the histone deacetylase inhibitor is depsipeptide. Depsipeptide may be administered to a patient by continuous i.v. infusion for at least 4 hours per day for a week, at a dose optionally ranging from 2-100 mg/m², optionally ranging from 5-50 mg/m², and optionally ranging from 5-15 mg/m². The treatment cycle may be 1 or 2 weeks per month. The formulation for the continuous i.v. infusion of depsipeptide may be formed by resuspending up to 5 mg/ml of depsipeptide in an ethanol based. The suspension is then further diluted in normal saline for iv administration.

Also optionally, the histone deacetylase inhibitor is phenylbutyrate (PB). PB may be administered to a patient by continuous i.v. infusion for at least 2-3 weeks at a dose optionally ranging from 100-2000 mg/m², optionally ranging from 250-1000 mg/m², and optionally ranging from 500-800 mg/m².

Also optionally, the histone deacetylase inhibitor is trichostatin A (TSA). TSA is administered to a patient by continuous i.v. infusion for 2-3 weeks at a dose optionally ranging from 100-2000 mg/m², optionally ranging from 250-1000 mg/m², and optionally ranging from 500-800 mg/m². Infusion can be continuous or preferably 1-12 hours per day for at least 1-4 weeks.

Optionally, the EZH2 antagonist is an EZH2 antibody. The EZH2 antibody may be administered to a patient by continuous i.v. fusion for at least 4 hours per day for a week, at a dose optionally ranging from 2-100 mg/m², optionally ranging from 5-50 mg/m², and optionally ranging from 5-15 mg/m².

Also optionally, the EZH2 antagonist is an EZH2 antisense nucleic acid. The EZH2 antisense nucleic acid may be administered to a patient using any methods known in the art for the introduction of an expression vector into cells, including but not limited to electroporation, cell fusion, DEAE-dextran mediated transfection, calcium phosphate-mediated transfection, infection with a viral vector, microinjection, lipofectin-mediated transfection, liposome delivery, and particle bombardment techniques, including various procedures for “naked DNA” delivery.

According to any of the above embodiments, after the treatment with the DNA methylation inhibitor (e.g., decitabine) or the histone deacetylase inhibitor, further treatment may include EZH2 antagonists, antineoplastic agents, surgery procedures, hormonal therapy and/or radiation.

Owing to the sensitizing effects of the combination therapy on the cells to apoptosis, the dosage of antineoplastic agents used for the treatment may be lower than that used in a conventional cancer treatment regimen.

Surgery procedures may include radical retropubic prostatectomy, radical perineal prostatectomy, and transurethral resection of the prostate (“TURP”). Radical retropubic prostatectomy involves the removal of the entire prostate and nearby lymph nodes though an incision in the abdomen. Radical perineal prostatectomy involves the removal of the entire prostate through an incision between the scrotum and the anus. Nearby lymph nodes are sometimes removed through a separate incision in the abdomen. A TURP involves the removal of part of the prostate with an instrument that is inserted through the urethra. The cancer is cut from the prostate by electricity passing through a small wire loop on the end of the instrument. This method is usually used mainly to remove tissue that blocks urine flow. A separate surgical procedure to remove lymph nodes may also be necessary if the disease has spread to other parts of the body.

Hormonal therapy can be achieved by orchiectomy or other drugs such as lutenizing hormone-releasing hormone (LH-RH) agonists, antiandrogens, and drugs that prevent the adrenal glands from making androgens. Orchiectomy is a surgical procedure to remove the testicles which are the main source of male hormones. LH-RH agonists prevent the testicles from producing testosterone. Three such drugs include leuprolide, goserelin, and buserein. Examples of Antiandrogens include flutamide and bicalutamide. Drugs that can prevent the adrenal glands from making androgens include ketoconazole and aminoglutethimide.

### BRIEF DESCRIPTION OF THE FIGURES

FIG. 1 illustrates chemical structures for 5-azacytidine and 5-aza-2′-deoxycytidine (“decitabine”).

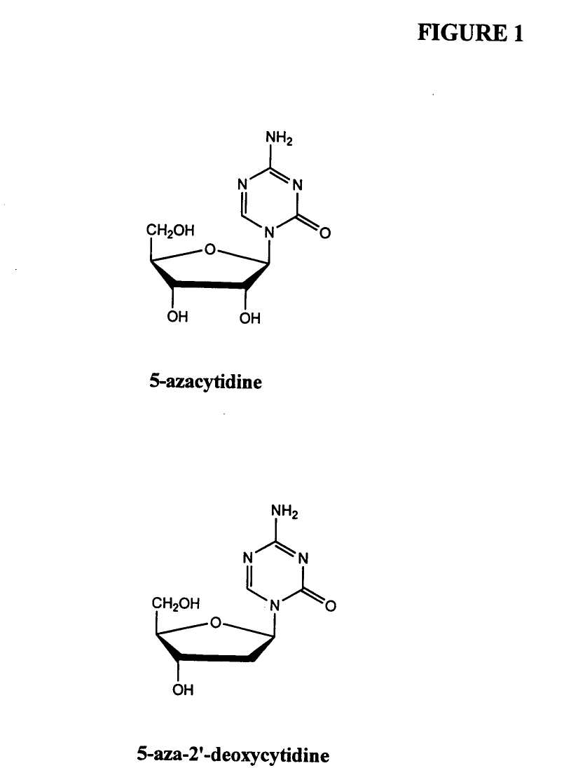

FIG. 2 illustrates chemical structures for some histone deacetylase inhibitors.

### DETAILED DESCRIPTION OF THE INVENTION

The present invention provides an innovative approach to the treatment of diseases or disorders, in particular, diseases associated with EZH2 over-expression. By exploiting the role EZH2 plays in DNA methylation, histone deacetylation, and transcription silencing of genes, the invention herein employs a DNA methylation inhibitor and/or a histone deacetylase inhibitor, optionally in combination with an EZH2 antagonist and/or an antineoplastic agent, to specifically target diseases associated with EZH2 over-expression, such as late stage prostate cancer. It is believed that genes which play critical roles in tumor suppression but are suppressed via the EZH2 signal transduction pathway may be reactivated through inhibition of EZH2 activities directly and/or indirectly. The treatment regimens provided in the present invention should have a higher therapeutic efficacy than current cancer therapy owing to the synergism resulted from a combination of agents targeting various different players in the EZH2 pathway.

1. EZH2 Expression

During embryonic development, many different cell types may arise from a single fertilized egg. Once a cell establishes its specific differentiation status, it requires a cellular memory system to allow the maintenance of proper and stably inherited gene expression pattern. See Sewalt et al. (2002), Mol. Cell Biol. 22(15): 5539-5553. The Polycomb-group (PcG) and Trithorax-group (TrxG) protein complexes are part of such cellular memory system. Generally, PcG proteins play an important role in maintaining genes silent, while TrxG proteins maintain genes in an active state of expression. Together, PcG and TrxG maintain gene expression patterns established during embryogenesis through many cell divisions. A unique protein that has both PcG and TrxG functionalities is EZH2 which is a mammalian homolog of the enhancer of zeste E(z) gene. Sewalt (1998) Mol. Cell Biol. 18(6): 3586-3595.

The EED/EZH2 PcG complex, both in humans and Drosophila, interacts with histone deacetylase (HDAC) and histone lysine methyltransferase (HMTase), both of which are associated with DNA methylation. Johan van der Vlag & Arie P. Otte, (1999) Nature Genetics 23:474-478; Cao, R. et al., (2002) Science 298: 1039-1043. As HDAC proteins generally do not interact with other vertebrate PcG proteins, the interaction between EED/EZH2 and HDAC is highly specific. See Johan van de Vlag & Arie P. Otte, supra. It has been demonstrated that deacetylation is mediated by EED both in vitro and in vivo. Id. As a result of histone deacetylation, the histones become more positively charged thus binding tighter to negatively charged DNA molecules. This increase in binding affinity between DNA and histones represses transcription of a cohort of genes associated with cell cycle, cell differentiation and apoptosis by changing the chromatin structure.

The EED/EZH2 complex has also been shown to interact with histone methyltransferase to selectively methylate histone H3 at lysine 27 (H3-K27). Cao, R. et al., (2002) Science 298: 1039-1043. Specifically, EZH2 interacts with histone lysine methyltransferase via a SET domain that is a signature motif for all known histone lysine methyltransferase, except H3-K79. As a result of this interaction, the histone lysine methyltransferase transfers a methyl group from a methyl donor S-adenosyl-L-methionine (SAM) to the narrow SET domain channel where the lysine awaits. The resulting methylated H3 histone may recruit PRC1 complex, which leads to the formation of a relaxed chromosome state leading to an increase of transcription activity. Cao, R. et al., (2002) Science 298: 1039-1043.

The methylation of lysine residues and acetylation of histones play a pivotal role in the regulation of chromatin structure, gene expression and DNA methylation. For example, it has been shown that DNA methyltransferase Dnmt1, and several methyl-CpG binding proteins, MeCP2, MBD2, MBD3, all associate with or recruit histone deacetylase which in turn repress transcription. In addition, methyl-binding proteins (MBDs) not only recruit histone deacetylase proteins but also are direct transcriptional repressors.

According to the present invention, to ameliorate the adverse effects of EZH2-mediated gene silencing, a DNA methylation inhibitor (e.g., decitabine) and/or a histone deacetylation inhibitor (e.g., phenylbutyrate) can be used to target key players along the EZH2 signal transduction pathway. Further, since the EED/EZH2 complex is associated with both histone methyltransferase activity and histone deacetylase activity, the administration of EZH2 antagonists may also contribute indirectly to the reactivation of transcription of repressed genes.

This inventive approach can be applied to a broad area of therapeutic treatment of diseases associated with EZH2 over-expression, and in particular cancer. EZH2 has been identified as being overly expressed in hormone-refractory, metastatic prostate cancer. See Varambally et al. (2002) Nature 419:624-629. Over-expression of EZH2 mRNA and EZH2 protein is also detected in localized prostate cancer that exhibits poorer diagnosis than in indolent prostate cancer, and in some forms of blood cancers (e.g., B-cell non-Hodgkin's lymphoma). See Folkert J. van Kemenade et al. (2002) Blood 97(12): 3896-3901. Other cell lines that show an increased level of EZH2 include those of the spleen, ovary and small intestine. Sewalt et al. (1998) Mol. Cell. Biol. 18(6): 3586-3595. By inhibiting EZH2-mediated DNA methylation and/or histone deacetylation, diseases associated with EZH2 over-expression can be treated more efficaciously than by traditional cancer therapy.

Prior to, during, or post treatment, EZH2 expression can serve as a marker to detect the presence and severity (stage) of disorders associated with over-expression of EZH2, especially prostate cancer and non-Hodgkin's lymphoma. EZH2 expression can also serve as a marker to distinguish early-stage from late-stage of a disease (e.g., benign prostate cancer and hormone-refractory, metastatic prostate cancer). Based on the levels of EZH2 expression, subpopulations of patients at a particular stage of cancer can be selected and treated with the therapy provided in the present invention.

2. EZH2 Antagonists

EZH2-mediated gene silencing can be inhibited by EZH2 antagonists. EZH2 antagonists are agents that diminish or interfere with EZH2's expression and/or activity. EZH2 antagonists can be used in diagnosis, prognosis and treatment of conditions associated with EZH2 over-expression, e.g., prostate cancer and non-Hodgkin's lymphoma. EZH2 antagonists include, for example, antisense nucleic acids that hybridize under high stringency conditions to EZH2 DNA or RNA, nucleic acids that form triple-helix formations with EZH2 DNA or RNA, small interfering RNA (siRNA) of EZH2, ribozymes, antibodies, fusion proteins, DNA binding proteins and small and large organic and inorganic molecules, or mimetics thereof.

Antibodies that specifically bind EZH2 gene products, include, for example, polyclonal antibodies, monoclonal antibodies, humanized or chimeric antibodies, single chain antibodies, FAb fragments, F(ab′)₂ fragments, fragments produced by a FAb expression library, anti-idiotypic (anti-Id) antibodies and epitope-binding fragments.

Polyclonal antibodies against EZH2 gene products can be prepared by immunizing a suitable subject (e.g., goats, rabbits, rats, mice, humans, etc.) with a desired immunogen, e.g., an EZH2 polypeptide or fragment thereof. The antibody titer in the immunized subject can be monitored over time using standard techniques, such as an enzyme linked immunosorbent assay (ELISA). If desired, the antibody molecules can be isolated from the mammal (e.g., from the blood) and further purified by well-known techniques, such as protein A chromatography to obtain the IgG fraction.

At an appropriate time after immunization, e.g., when the antibody titers are highest, antibody producing cells can be obtained from the subject and used to prepare monoclonal antibodies using standard techniques, including the hybridoma technique originally described by Kohler and Milstein (1975) Nature, 256:495-497, the human B cell hybridoma technique described in Kozbor et al. (1983) Immunol. Today, 4:72, the EBV-hybridoma technique described in Cole et al. (1985), Monoclonal Antibodies and Cancer Therapy, Alan R Liss, Inc., pp. 77-96 or trioma techniques. Technologies for producing hybridomas are well known. See generally Coligan et al. “Current Protocols in Immunology,” (eds.) (John Wiley & Sons, Inc., New York, N.Y. 1994). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds polypeptide of interest, e.g. EZH2.

Other means for generating monoclonal antibodies include screening a recombinant combinatorial immunoglobulin library (e.g., an antibody phage display library) with an EZH2 polypeptide to isolate immunoglobulin library members that bind the polypeptide. Kits for generating and screening phage display libraries are commercially available. See the Pharmacia Recombinant Phage Antibody System, Catalog No. 27-9400-01; and the Stratagene SurjZAPTM Phage Display Kit, Catalog No. 240612). Additionally, examples of methods and reagents particularly amenable for generating and screening antibody display libraries can be found in, for example, Huse et al. (1989) Science 246:1275-1281; and Griffiths et al. (1993) EMBO J. 12:725-734.

In a preferred embodiment, monoclonal antibodies are chimeric and humanized. Humanized monoclonal antibodies can be obtained using standard recombinant DNA techniques in which the variable region genes of a rodent antibody are cloned into a mammalian expression vector containing the appropriate human light change and heavy chain region genes. Such that the resulting chimeric monoclonal antibodies have the antigen-binding capacity from the variable region of, for example, a rodent, but should be significantly less immunogenic because of the human light and heavy chain regions. See, e.g., Surender K. Vaswani, Ann. Allergy Asthma. Immunol. (1998); 81:105-119.

The antibodies herein can be used to detect the level of expression EZH2 (e.g., in a cellular lysate, cell supernatant, or tissue sample) in order to evaluate, for example, severity of disease and effectiveness of treatment. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H. The antibodies herein can also be used as EZH2 antagonists, to diminish or inhibit the expression of EZH2.

In addition to antibodies, antisense nucleic acids, can also be used to detect or inactivate EZH2 by specifically hybridizing to EZH2 DNA or RNA. Specific hybridization refers to the binding, duplexing, or hybridizing of a molecule preferentially to a particular nucleotide sequence when that sequence is present in a complex mixture (e.g., total cellular) DNA or RNA. Hybridization conditions required may vary depending on the buffers used, length of nucleic acids, etc. Stringency conditions for hybridization refers to the incubation and wash conditions (e.g., conditions of temperature and buffer concentration), which permit hybridization of a particular nucleic acid to a second nucleic acid; the first nucleic acid may be perfectly (i.e. 100%) complementary to the second, or the first and second may share some degree of complementarity which is less than perfect (e.g., more than 70%, 75%, 85%, or 95%).

The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary hybridization conditions are described in Ausubel, et al., “Current Protocols in Molecular Biology” (John Wiley & Sons, 1998). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in TM of ˜17° C. Using these guidelines, the washing temperature can be determined empirically for high, moderate or low stringency, depending on the level of mismatch sought. For example, a low stringency wash can comprise washing in a solution containing 0.2×SSC/0.1% SDS for 10 min at room temperature; a moderate stringency wash can comprise washing in a pre-warmed solution (42° C.) solution containing 0.2×SSC/0.1% SDS for 15 min at 42° C; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleotlytic cleavage. Ribozyme can be designed to be complementary to the EZH2 MRNA sequence. Ribozyme can further include a well known catalytic sequence responsible for MRNA cleavage. See Cech, et al. U.S. Pat. No. 5,093,246. Ribozymes can also include an engineered hammerhead motif that specifically and efficiently catalyzes endonucleolytic cleavage of RNA sequences encoding EZH2 proteins. Such ribozymes can be used to inactivate EZH2 mRNA, thus blocking EZH2 translation, expression and activity.

In addition, nucleic acids can be designed to form a triple helix with EZH2 DNA or RNA, which can be used to inhibit transcription of EZH2 gene. Triple-helix forming nucleic acid should be single stranded and composed of deoxynucleotides. The base composition of these nucleic acids must be designed to promote triple helix formation via Hoogsteen base pairing rules, which generally require sizeable stretches of either purines or pyrimidines on one strand of the duplex. In addition, insertion of a double-stranded EZH2 RNA can effectively cause destruction of EZH2 mRNA by operating as small interfering RNA. The antisense nucleic acids, ribozymes (RNA and DNA) and triple helix molecules disclosed herein can be prepared by standard methods known in the art for the synthesis of DNA and RNA molecules.

In another embodiment, polypeptides that bind to or interact with EZH2 thus inhibiting EZH2 activity are disclosed. Such polypeptides may be identified using a yeast two-hybrid system, such as that described in Fields, S. and Song, O., (1989) Nature 340:245-246. The yeast two-hybrid system utilizes two vectors: one comprising a DNA binding domain and the other a transcription activation domain. The domains are fused to two different proteins. If the two proteins interact with one another, transcription can occur. Transcription of specific markers (e.g., nutritional markers such as His and Ade, or color markers such as lacZ) is used to identify the presence of such interaction. Incubation of yeast containing the two vectors under appropriate conditions (e.g., mating conditions such as used in the Matchmaker system from Clontech) allows for the identification of colonies that express the markers of interest. Such colonies can be examined to identify the polypeptides that interact with EZH2 and are antagonist to EZH2 activity.

EZH2 antagonists may also comprise of small and large organic and inorganic molecules that inhibit EZH2 expression and/or activity. Small molecules are preferred because such molecules are more readily absorbed after oral administration and have fewer potential antigenic determinants. Small molecules are also more likely to cross the blood brain barrier than larger protein-based pharmaceuticals. Non-peptide agents or small molecule libraries are generally prepared by a synthetic approach, but recent advances in biosynthetic methods using enzymes may enable one to prepare chemical libraries that are otherwise difficult to synthesize chemically. Small molecule libraries can also be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, U.K.), and Asinex (Moscow, Russia). These small molecule libraries can be used for screening in a high throughput manner to identify one or more agents. For example, a high throughput screening assay for small molecules that was disclosed in Stockwell, B. R. et al., Chem. & Bio., (1999) 6:71-83, is a miniaturized cell-based assay for monitoring biosynthetic processes such as DNA synthesis and post-translational processes.

Methods for screening small molecule libraries for candidate protein-binding molecules are well known in the art and may be employed to identify molecules that bind to EZH2 . Briefly, EZH2 protein may be immobilized on a substrate and a solution containing the small molecules is contacted with an EZH2 polypeptide under conditions that are permissive for binding. The substrate is then washed with a solution that substantially reflects physiological conditions to remove unbound or weakly bound small molecules. A second wash may then elute those compounds that are bound strongly to the immobilized polypeptide. Alternatively, the small molecules can be immobilized and a solution of EZH2 polypeptides can be contacted with the column, filter or other substrate. The ability of an EZH2 polypeptide to bind to a small molecule may be determined by labeling (e.g., radio-labeling or chemiluminescence). Small molecules that bind EZH2 can diminish or inhibit EZH2 activity and are therefore useful as EZH2 antagonists.

Any of the EZH2 antagonists disclosed herein can be administered in therapeutically effective amounts that are determined by standard clinical techniques. The precise dosage to be employed in a formulation depends in part on the method of administration, type of condition, seriousness of symptoms, age, sex, and body weight of patient, and other factors. Final dosage should be decided according to the judgment of a practitioner based upon each patient's circumstances. Cell-based or animal models can also be used to determine the precise dosages to be administered.

Generally, the LD₅₀ (the lethal dose to 50% of the population) and the ED₅₀ (the effective dose in 50% of the population) of a pharmaceutical composition can be determined using cell cultures or animal models following standard pharmaceutical procedures. The dose ratio of lethal and effective doses is the therapeutic index and is expressed as the ratio, LD₅₀/ED₅₀. Compounds that exhibit large therapeutic indices are preferred. Compounds that exhibit toxic side effects can also be used, but care should be taken to design a delivery system that targets such compounds to the site of affected tissue to minimize potential damage to uninfected cells.

When using cell culture to estimate the therapeutically effective dose, the dosage of such compounds lies preferably within a range of circulating concentrations that include the ED₅₀ with little or no toxicity. A dose can also be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography.

3. DNA Methylation and Inhibitors

As described above, DNA methylation that is affected directly and/or indirectly by EZH2 activities can be inhibited using a DNA methylation inhibitor, such as decitabine. Through inhibition of DNA methylation, transcription of genes that have been silenced or repressed by the EZH2 mechanisms, such as tumor suppressor genes, can be reestablished leading to effective inhibition of tumor growth and metastasis.

In mammalian cells, approximately 3-5% of the cytosine residues in genomic DNA are present as 5-methylcytosine. Ehrlich et al. (1982) Nucleic Acid Res. 10:2709-2721. This modification of cytosine takes place after DNA replication and is catalyzed by DNA methyltransferase (DNMTs) using S-adenosyl-methionine (SAM) as the methyl donor. (SAM may also interact with histone methyltransferase (HMTase), which can transfers a methyl group to specific lysine groups on H3. See Taewoo, K., et al. (2003) EMBO J. 22 (2): 292-303.)

Overall, DNA methylation induces decreased levels of chromatin acetylation by HDAC. See Eden S., et al. (1998) Nature 394:842. Thus, the link between DNA methylation and chromatin acetylation lies first in the recruitment to methylated cytosines a family of methyl-CpG binding domain proteins (MBDs), which are direct transcriptional repressors and can complex with transcriptional corepressors including histone deacetylases (HDACs). See Rountree M. R., (2001) Oncogene 2 (24): 3156-3165. Id. Second, DNMTs also directly repress transcription and associate with HDACs such that synergy between HDAC activity and DNMT is a key to tumorgenesis and tumor suppression. Id. Third, DNMTs utilizes SAM as a donor which may also be the methyl donor for HMTase, thus the more SAM interacts with DNMTs to increase DNA methylation, the less it is able to interact with HMTase resulting in deacetylated histones.

Approximately 70% to 80% of 5-methylcytosine residues are found in the CpG dinucleotide sequence. Bird (1986) Nature 321:209-213. However, when CpG are found in high density in mammalian genome, it is normally in non-methylated state. These high-density unmethylated CpG sequences are known as CpG islands. Unmethylated CpG islands are associated with housekeeping genes (or promoters thereof) that are always turned on and are resistant to methylation. Antequera and Bird (1999) Current Biology, 9:R661-667. This methylation of DNA has been proposed to play an important role in the control of expression of different genes in eukaryotic cells during embryonic development. For example, DNA methylation in the CpG islands of a promoter region of a gene is believed to interfere with the binding of transcription factors, thus suppressing the expression of the gene. This may be because 5-methylcytosine protrudes into the major groove of the DNA helix, which interferes with the binding of transcription factors. Consistent with this hypothesis, inhibition of DNA methylation has been found to induce differentiation in mammalian cells. Jones and Taylor (1980) Cell 20:85-93.

The methylated cytosine in DNA, 5-methylcytosine, can undergo spontaneous deamination to form thymine at a rate much higher than the deamination of cytosine to uracil. Shen et al. (1994) Nucleic Acid Res. 22:972-976. If the deamination of 5-methylcytosine is unrepaired, it will result in a C to T transition mutation. For example, many “hot spots” of DNA damages in the human p53 gene are associated with CpG to TpG transition mutations. Denissenko et al. (1997) Proc. Natl. Acad. Sci. USA 94:3893-1898.

Other than p53 gene, many tumor suppressor genes can also be inactivated by aberrant methylation of the CpG islands in their promoter regions. Many tumor-suppressors and other cancer-related genes have been found to be hypermethylated in human cancer cells and primary tumors. Examples of genes that participate in suppressing tumor growth and are silenced by aberrant methylation include, but are not limited to, tumor suppressors such as p15/INK4B (cyclin kinase inhibitor, p16/INK4A (cyclin kinase inhibitor), p73 (p53 homology), ARF/INK4A (regular level p53), Wilms tumor, von Hippel Lindau (VHL), retinoic acid receptor-β(RAR-β), estrogen receptor, androgen receptor, mammary-derived growth inhibitor hypermethylated in cancer (HIC1), and retinoblastoma (Rb); Invasion/metastasis suppressor such as E-cadherin, tissue inhibitor metalloproteinase-2 (TIMP-3), mts-1 and CD44; DNA repair/detoxify carcinogens such as methylguanine methyltransferase, hMLH1 (mismatch DNA repair), glutathione S-transferase, and BRCA-1; Angiogenesis inhibitors such as thrombospondin-1 (TSP-1) and TIMP3; and tumor antigens such as MAGE-1.

In particular, silencing of p16 is frequently associated with aberrant methylation in many different types of cancers. The p16/INK4A tumor suppressor gene codes for a constitutively expressed cyclin-dependent kinase inhibitor, which plays a vital role in the control of cell cycle by the cyclin D-Rb pathway. Hamel and Hanley-Hyde (1997) Cancer Invest. 15:143-152. P16 is located on chromosome 9p, a site that frequently undergoes loss of heterozygosity (LOH) in primary lung tumors. In these cancers, it is postulated that the mechanism responsible for the inactivation of the non-deleted allele is aberrant methylation. Indeed, for lung carcinoma cell lines that did not express p16, 48% showed signs of methylation of this gene. Otterson et al. (1995) Oncogene 11:1211-1216. About 26% of primary non-small cell lung tumors showed methylation of p16. Primary tumors of the breast and colon display 31% and 40% methylation of p16, respectively. Herman et al. (1995) Cancer Res. 55:4525-4530.

Aberrant methylation of retinoic acid receptors are also attributed to development of breast cancer, lung cancer, ovarian cancer, etc. Retinoic acid receptors are nuclear transcription factors that bind to retinoic acid responsive elements (RAREs) in DNA to activate gene expression. In particular, the putative tumor suppressor RAR-β gene is located at chromosome 3p24, a site that shows frequent loss of heterozygosity in breast cancer. Deng et al. (1996) Science 274:2057-2059. Transfection of RAR-β cDNA into some tumor cells induced terminal differentiation and reduced their tumorigenicity in nude mice. Caliaro et al. (1994) Int. J. Cancer 56:743-748; and Houle et al. (1993) Proc. Natl. Acad. Sci. USA 90:985-989. Lack of expression of the RAR-β gene has been reported for breast cancer and other types of cancer. Swisshelm et al. (1994) Cell Growth Differ. 5:133-141; and Crowe (1998) Cancer Res. 58:142-148. This reason for lack of expression of RAR-β gene is attributed to methylation of RAR-β gene. Indeed, methylation of RAR-β was detected in 43% of primary colon carcinomas and in 30% of primary breast carcinoma. Cote et al. (1998) Anti-Cancer Drugs 9:743-750; and Bovenzi et al. (1999) Anticancer Drugs 10:471-476.

Methylation of CpG islands in the 5′-region of the estrogen receptor gene has been found in multiple tumor types. Issa et al. (1994) J. Natl. Cancer Inst. 85:1235-1240. The lack of estrogen receptor expression is a common feature of hormone unresponsive breast cancers, even in the absent of gene mutation. Roodi et al. (1995) J. Natl. Cancer Inst. 87:446-451. About 25% of primary breast tumors that were estrogen receptor-negative displayed aberrant methylation at one site within this gene. Breast carcinoma cell lines that do not express the mRNA for the estrogen receptor displayed increased levels of DNA methyltransferase and extensive methylation of the promoter region for this gene. Ottaviano et al. (1994) Cancer Res. 54:2552-2555.

Methylation of human mismatch repair gene (hMLH-1) is also found in various tumors. Mismatch repair is used by the cell to increase the fidelity of DNA replication during cellular proliferation. Lack of this activity can result in mutation rates that are much higher than that observed in normal cells. Modrich and Lahue (1996) Annu. Rev. Biochem. 65:101-133. Methylation of the promoter region of the mismatch repair gene (hMLH-1) was shown to correlate with its lack of expression in primary colon tumors, whereas normal adjacent tissue and colon tumors the expressed this gene did not show signs of its methylation. Kane et al. (1997) Cancer Res. 57:808-811.

The molecular mechanisms by which aberrant methylation of DNA takes place during tumorigenesis are not clear. It is possible that the DNA methyltransferase makes mistakes by methylating CpG islands in the nascent strand of DNA without a complementary methylated CpG in the parental strand. It is also possible that aberrant methylation may be due to the removal of CpG binding proteins that “protect” these sites from being methylated. Whatever the mechanism, the frequency of aberrant methylation is a rare event in normal mammalian cells.

Decitabine, 5-aza-2′-deoxycytidine, is an antagonist of its related natural nucleoside, deoxycytidine. The only structural difference between these two compounds is the presence of a nitrogen at position 5 of the cytosine ring in decitabine as compared to a carbon at this position for deoxycytidine. Two isomeric forms of decitabine can be distinguished. The β-anomer is the active form. The modes of decomposition of decitabine in aqueous solution are (a) conversion of the active b-anomer to the inactive β-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent forming of guanidine compounds (Kissinger and Stemm (1986) J. Chromat. 353:309-318).

Decitabine possesses multiple pharmacological characteristics. At a molecular level, it is capable of specifically inhibiting DNA methylation and cell growth at S phase. At a cellular level, decitabine can induce cell differentiation and exert hematological toxicity. Despite having a short half life in vivo, decitabine has excellent tissue distribution.

The most prominent function of decitabine is its ability to specifically and potently inhibit DNA methylation by specifically binding to DNA methyltransferase, thus blocking methylation. As described above for methylation of cytosine in CpG islands as an example, methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) Pharmacol Ther. 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes can be prevented.

According to the present invention, the inventor take advantage of the ability of DNA methylation inhibitors, such as decitabine, reactivate the tumor suppressor genes silenced by aberrant methylation. By reducing methylation, these agents can render more effective anti-neoplastic agents whose pharmaceutical activities are adversely affected by methylation in vivo.

4. Histone Deacetylation and Inhibitors

Histone deacetylation that is affected directly or indirectly by EZH2 activities can also be inhibited by using a histone deacetylase inhibitor, such as phenylbutyrate. Through inhibition of the EZH2-mediated histone deacetylation, transcription of genes that have been silenced or repressed, such as tumor suppressor genes, can be reestablished, leading to effective inhibition of tumor growth and metastasis.

The DNA of all chromosomes is packaged into a compact structure with the aid of specialized proteins. In eucaryotes, these special DNA-binding proteins are divided into two general classes: histones and nonhistone chromosomal proteins. Together, the nuclear DNA and DNA-binding proteins make up a complex known as a chromatin. Histones are unique to eucaryotes and the principal structural proteins of eucaryotic chromosomes. They are present in such enormous quantities that their total mass in chromatin is roughly equal to that of the DNA.

There are five types of histones identified in chromatin: H1, H2A, H2B, H3, and H4. These five types fall into two groups: the nucleosomal histones and the H1 histones. The nucleosomal histones (H2A, H2B, H3, and H4) are small proteins (102-105 amino acids) responsible for coiling the DNA into nucleosomes. The H1 histones are larger (containing about 220 amino acids). They occur in chromatin in about half the amount of the other types of histones and appear to lie on the outer portion of the nucleosome.

Histones play a crucial part in packing of chromosomal DNA and activation of genes within. Histones pack the very long helix of DNA in each chromosome in an orderly way into a nucleus only a few micrometers in diameters. The role of histones in DNA folding is important in that the manner in which a region of the genome is packaged into chromatin in a particular cell influences the activity of the genes the region contains.

Chromatin structure of transcribed genes is less condensed than that of the untranscribed or silenced genes. Studies have shown that transcriptionally active chromatin is biochemically distinct from that of the inactive chromatin. The analysis of the chromosomal proteins in the active chromatin suggested the following biophysical and biochemical characteristics: 1) Histone H1 seems to be less tightly bound to at least some active chromatin; 2) the four nucleosomal histones appear to be unusually highly acetylated when compared with the same histones in inactive chromatin; and 3) the nucleosomal histone H2B in active chromatin appears to be less phosphorylated than it is in inactive chromatin. These changes in chromatin features play an important part in uncoiling the chromatin of active genes, helping to make the DNA available as a template for RNA synthesis during transcription of the gene.

In particular, acetylation and deacetylase of histone plays important roles in regulation of gene expression. Acetylation of the lysine or arginine residues at the N-terminus of histone proteins removes positive charges, thereby reducing the affinity between histones and DNA. This makes it easier for RNA polymerase and transcription factors to access the promoter region and enhances transcription. Conversely, deacetylase of histones restores positive charge to the amino acids and results in tighter binding of histones to the negatively charged phosphate backbone of DNA. Such a condensed chromatin DNA conformation is relatively inaccessible to the transcription machinery and thus the genes in the condensed area are not expressed, i.e. silenced. It has been demonstrated that chromatin fractions enriched in actively transcribed genes are also enriched in highly acetylated core histones, whereas silent genes are associated with nucleosomes with a low level of acetylation. Kouzarides (1999) Curr. Opin Genet. Dev. 9:40-48.

The amount histone acetylation is controlled by opposing activities of two types of enzymes, histone acetyl transferases (HATs) and histone deacetylases (HDACs). Substrates for these enzymes include the lysine residues located in the amino-terminal tails of histones H2A, and H2B, H3, H4. These residues are acetylated by HATs and deacetylated by HDACs such that to activate these genes silenced by deacetylase of histones, the activity of HDACs should be inhibited. With the inhibition of HDACs, histones are acetylated and bound less tightly to the DNA, opening the DNA conformation to transcription of specific genes.

EZH2/EED proteins interact with HDACs both in vivo and in vitro in mediating gene suppression. See Johan can der Vlag & Arie P. Otte, (1999) Nature Genetics 23:474-478. Thus, an increased level of EZH2 expression indicates a greater amount of tumor gene suppression via HDAC.

In addition to deacetylation of histones, HDACs may also regulate gene expression by deacetylating transcription factors, such as p53 (a tumor suppressor gene), GATA-1, TFIIE, and TFIIF. Gu and Roeder (1997) Cell 90:595-606; and Boyes et al. (1998) Nature 396:594-598. HDACs also participate in cell cycle regulation, for example, by transcription repression of RB tumor suppressor proteins. Brehm et al. (1998) Nature 391:597-601. Thus, inhibition of HDACs should activate expression of tumor-suppressor genes such as p53 and RB and as a result promote cell growth arrest, differentiation and apoptosis induced by these genes.

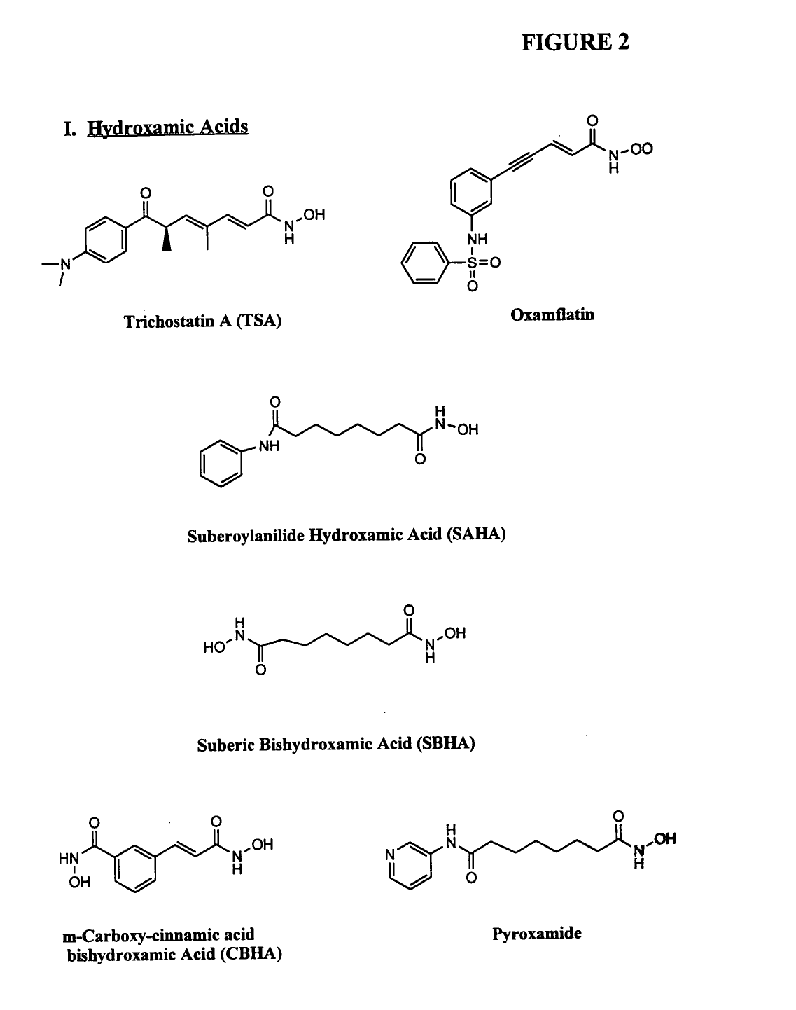

Inhibitors of HDACs include, but are not limited to, the following structural classes: hydroxamic acids, cyclic peptides, benzamides, and short-chain fatty acids. In a particular embodiment, trichostatin A can relieve histone deacetylation mediated by EED. See Johan can der Vlag & Arie P. Otte, (1999) Nature Genetics 23:474-478. Chemical structures for some of these HDAC inhibitors are shown in FIG. 2.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), and pyroxamide. TSA was isolated as an antifungi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature—a polar hydroxamic terminal linked through a hydrophobic methylene spacer (e.g., six carbon at length) to another polar site that is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and FR901228. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activity and inhibits HDAC activity at nanomolar concentrations. Darkin-Rattray et al. (1996) Proc. Natl. Acad. Sci. USA. 93;13143-13147. FR901228 is a depsipeptide that is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597. Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor of the present invention.

5. Anti-Neoplastic Agents

A wide variety of antineoplastic agents may be used in conjunction with the combination of the DNA methylation inhibitor and/or the histone deacetylase inhibitor for treating neoplastic diseases associated with EZH2 over-expression, such as prostate cancer. Such antineoplastic agents can be categorized as: antibiotic agents, antimetabolic agents, plant derived agents, and biologic agents.

Antibiotic agents are a group of anticancer drugs that are produced in a manner similar to antibiotics by a modification of natural products. Examples of antibiotic agents include, but are not limited to, anthracyclines (e.g., doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and form an activated complex, which then binds to bases of DNA, causing strand scissions and cell death. Such a combination therapy may have therapeutic synergistic effects on cancer and reduce sides affects associated with these chemotherapeutic agents.

Other forms of antibodies include monoclonal antibodies. Monoclonal antibodies against tumor are antibodies elicited against antigens expressed by tumors, preferably tumor-specific antigens. Examples of monoclonal antibodies for cancer therapy include, but are not limited to, HERCEPTIN® (Trastruzumab), RITUXAN® (Rituximab), PANOREX® (edrecolomab), ZEVALIN® (ibritumomab yiuxetan), MYLOTARGT® (gemtuzumab ozogamicin), and CAMPATH® (alemtuzumab).

HERCEPTIN(® (Trastruzumab) is a monoclonal antibody raised against human epidermal growth factor receptor 2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including a DNA methylation inhibitor and HERCEPTIN® may have therapeutic synergistic effects on tumors, especially on metastatic cancers.

RITUXAN® (Rituximab) was elicited against CD20 on lymphoma cells and selectively depleted normal and malignant CD20⁺ pre-B and mature B cells. RITUXAN® is used as single agent for the treatment of patients with relapsed or refractory low-grade or follicular, CD20⁺, B cell non-Hodgkin's lymphoma. Combination therapy including a DNA methylation inhibitor and RITUXAN® may have therapeutic synergistic effects not only on lymphoma, but also on other forms or types of malignant tumors.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Such a combination therapy may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), water soluble or insoluble camptothecin (e.g., 20(S)-camptothecin, 9-nitro-camptothecin, 9-nitro-camptothecin, and topotecan), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Camptothecin is believed to be a potent inhibitor of the nuclear enzyme DNA topoisomerase I (topo-I), which is responsible for “relaxation” of supercoiled double-stranded DNA by creating single-stranded breaks through which another DNA strand can pass during transcription. Topo-I reseals the break allowing DNA replication to occur. Inhibition of topo-I leads to the formation of stable DNA-topoisomerase complexes, with eventual formation of irreversible double-stranded DNA breaks, leading to apoptosis and/or other forms of cell death. Podophyllotoxins such as etoposide are believed to interfere with DNA synthesis by interacting with topoisomerase II, leading to DNA strand scission. Such a combination therapy may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including a DNA methylation inhibitor, a histone deacetylase inhibitor and the biologic agent may have therapeutic synergistic effects on cancer, enhance the patient's immune responses to tumorigenic signals, and reduce potential sides affects associated with this biologic agent.

Cytokines possess profound immunomodulatory activity. Some cytokines such as interleukin-2 (IL-2, aldesleukin) and interferon-α (IFN-α) demonstrate antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with a DNA methylation inhibitor include, but are not limited to, interleukin 2 (IL-2), and interleukin 4 (IL-4), interleukin 12 (IL-12).

Interferon-α includes more than 23 related subtypes with overlapping activities, all of the IFN-α subtypes within the scope of the present invention. IFN-α has demonstrated activity against many solid and hematologic malignancies, the later appearing to be particularly sensitive. Examples of interferons that may be used in conjunction with a DNA methylation inhibitor include, but are not limited to, interferon-α, interferon-β (fibroblast interferon) and interferon-γ (fibroblast interferon).

Other cytokines that may be used in conjunction with a DNA methylation inhibitor include those cytokines that exert profound effects on hematopoiesis and immune functions. Examples of such cytokines include, but are not limited to erythropoietin (epoietin-α), granulocyte-CSF (filgrastin), and granulocyte, macrophage-CSF (sargramostim). These cytokines may be used in conjunction with a DNA methylation inhibitor to reduce chemotherapy-induced myelopoietic toxicity.

Immuno-modulating agents other than cytokines may also be used in conjunction with a DNA methylation inhibitor to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

6. Indications for Treatment

Preferable indications that may be treated using the methods of the present invention include those involving undesirable or uncontrolled cell proliferation that is manifested by over-expression of EZH2. Such indications may include benign tumors, various types of cancers such as primary tumors and tumor metastasis, hematologic disorders (e.g., leukemia, myelodysplastic syndrome and sickle cell anemia), restenosis (e.g., coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor, cells become undifferentiated, stop responding to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, B-cell non-Hodgkin's lymphomas, malignant melanomas, epidermoid carcinomas, and other carcinomas and sarcomas.

Examples of B-cell non-Hodgkin's lymphomas include, but are not limited to, small lymphocytic lymphoma, follicular lymphoma, large B-cell lymphoma, mantle-cell lymphoma, and Burkitt lymphoma. Diagnostic of non-Hodgkin's lymphoma is usually made using lymph node biopsy and extranodal biopsies as necessary and hematopathology screening.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia.

Acute promyelocytic leukemia (APML) represents a distinct subgroup of AML. This subtype is characterized by promyelocytic blasts containing the 15;17 chromosomal translocation. This translocation leads to the generation of the fusion transcript comprised of the retinoic acid receptor and a sequence PML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

Prostate cancer may result from hereditary or environmental factors or both. Treatment for prostate cancer often depends on the severity of form of the disease. If the prostate cancer is benign, treatment may be as mild as constant monitoring. If on the other hand, the prostate cancer is metastatic or hormone refractory, the proscribed treatment may comprise of surgery, chemotherapy or both.

7. Formulations and Routes of Administration

A wide variety of delivery methods and formulations for different delivery methods may be used in the combination therapies of the present invention.

The inventive combination of therapeutic agents may be administered as compositions that comprise the inventive combination of therapeutic agents. Such compositions may include, in addition to the inventive combination of therapeutic agents, conventional pharmaceutical excipients, and other conventional, pharmaceutically inactive agents. Additionally, the compositions may include active agents in addition to the inventive combination of therapeutic agents. These additional active agents may include additional compounds according to the invention, or one or more other pharmaceutically active agents. In preferable embodiments, the inventive compositions will contain the active agents, including the inventive combination of therapeutic agents, in an amount effective to treat an indication of interest.

The inventive combination of therapeutic agents and/or compositions may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or co-administered in slow release dosage forms.

The inventive combination of therapeutic agents and compositions may be administered by a variety of routes, and may be administered or co-administered in any conventional dosage form. Coadministration in the context of this invention is defined to mean the administration of more than one therapeutic in the course of a coordinated treatment to achieve an improved clinical outcome. Such coadministration may also be coextensive, that is, occurring during overlapping periods of time. For example, the DNA methylation inhibitor may be administered to a patient before, concomitantly, or after the histone deacetylase inhibitor is administered. In preferred embodiments, the patient may be pretreated with the DNA methylation inhibitor (e.g., decitabine) and then treated with the histone deacetylase inhibitor (e.g., depsipeptide).

In any of the embodiment herein, an EZH2 antagonist may be administered to a patient before, concomitantly, or after the histone deacetylase inhibitor or DNA methylation inhibitor is administered. In preferred embodiments, the EZH2 antagonist is administered prior to treatment with the DNA methylation inhibitor or the histone deacetylase inhibitor.

Furthermore, in any of the embodiments herein, an anti-neoplastic agent may be administered to a patient suffering from a disease related to over-expression of EZH2. The anti-neoplastic agent may be administered to a patient in need of such treatment before, concomitantly, or after the histone deacetylase inhibitor or DNA methylation inhibitor is administered. The anti-neoplastic agent is preferably administered after the EZH2 antagonist is administered.

Amounts of the inventive combination of therapeutic agents can vary, according to determinations made by one of skill, but preferably are in amounts effective to create a cytotoxic or cytostatic effect at the desired site. Preferably, these total amounts are less than the total amount adding the maximum tolerated dose for each of the DNA methylation inhibitor and the histone deacetylase inhibitor, and more preferably less than the total amount added for individual administration of each of these inhibitors.

For the slow-release dosage form, appropriate release times can vary, but preferably should last from about 1 hour to about 6 months, most preferably from about 1 week to about 4 weeks. Formulations including the inventive combination of therapeutic agents and/or composition can vary, as determinable by one of skill, according to the particular situation, and as generally taught herein.

In any of the embodiment herein, decitabine is the preferred DNA methylation inhibitor. Decitabine may be supplied as sterile powder for injection, together with buffering salt such as potassium dihydrogen and pH modifier such as sodium hydroxide. This formulation is preferably stored at 2-8° C., which should keep the drug stable for at least 2 years. This powder formulation may be reconstituted with 10 ml of sterile water for injection. This solution may be further diluted with infusion fluid known in the art, such as 0.9% sodium chloride injection, 5% dextrose injection and lactated ringer's injection. It is preferred that the reconstituted and diluted solutions be used within 4-6 hours for delivery of maximum potency.

In a preferred embodiment, decitabine is administrated to a patient by injection, such as bolus i.v. injection, continuous i.v. infusion and i.v. infusion over 1 hour. For example, decitabine may be administered into the patient via a 1-24 hour i.v. infusion per day for 3-5 days per treatment cycle at a dose preferably ranging from 1-100 mg/m², more preferably ranging from 2-50 mg/m², and most preferably from 5-20 mg/m². The preferred dosage below 50 mg/m² for decitabine is considered to be much lower than that used in conventional chemotherapy for cancer. By using such a low dose of decitabine, transcriptional activity of genes silenced in the cancer cells can be activated to trigger downstream signal transduction for cell growth arrest, differentiation and apoptosis that eventually results death of these cancer cells. This low dosage, however, should have less systemic cytotoxic effect on normal cells, and thus have less side effects on the patient being treated.

For the histone deacetylase inhibitor, the dosage form depends on the type of compound used as the inhibitor. For example, depsipeptide may be formulated for i.v. infusion. In one embodiment, depsipeptide is administered to a patient by continuous i.v. infusion for at least 4 hours per day for a week at a dose preferably ranging from 2-100 mg/m², more preferably at a dose ranging from 5-50 mg/m², and most preferably at a dose ranging from 5-15 mg/m². The treatment cycle may be 1 or 2 weeks per month or longer if necessary.

In another embodiment, phenylbutyrate (PB) is administered to a patient by continuous i.v. infusion at a dose preferably ranging from 100-2000 mg/m², more preferably at a dose ranging from 250-1000 mg/m², and most preferably at a dose ranging from 500-800 mg/m². PB infusion can be continuous or at least 1-12 hours per day. The infusion regimen usually lasts for at least 2-3 weeks.

In another embodiment, arginine butyrate is administered to a patient by continuous i.v. infusion at a dose preferably ranging from 100-2000 mg/m², more preferably at a dose ranging from 250-1000 mg/m², and most preferably at a dose ranging from 500-800 mg/m². For example, arginine butyrate may be administered at a dose between 250-1000 mg/m² as a 6-12 hour i.v. infusion for 4 days every 2 weeks.

In another embodiment, trichostatin A (TSA) is administered to a patient by continuous i.v. infusion at a dose preferably ranging from 100-2000 mg/m², more preferably at a dose ranging from 250-1000 mg/m², and most preferably at a dose ranging from 500-800 mg/m². TSA infusion may be administered as a continuous infusion or at least 1-12 hour per day of i.v. infusion for at least 1-4 weeks.

Dosages of EZH2 antagonists will depend on the type of compound used. For example, EZH2 monoclonal antibodies can be administered at level similar to those of other anti-neoplastic agents monoclonal antibodies (e.g., RITUXIN®, HERCEPTIN®, etc.) EZH2 antisense, ribozyme, and triple-helix treatments are administered preferably locally and at concentrations derived from techniques known in the art.

In preferred embodiment, EZH2 antibodies are administered after administration of decitabine to the patient. In another preferred embodiment, depsipeptide or TSA are administered after administration of decitabine to the patient. This clinical regimen is designed to enhance efficacy of the combination therapy by sensitizing the cancers to apoptosis signals through inhibition of methylation and then triggering cell death by EZH2 or depsipeptide-induced apoptosis mechanism.

After the treatment with the DNA methylation inhibitor and histone deacetylase inhibitor, the patient may be further treated with various anticancer agents described above. Owing to the sensitizing effects of the combination therapy on the cells to apoptosis, the dosage of anticancer agents used for the treatment may be lower than that used in a convention cancer treatment regimen. Thus, a better clinical outcome may be achieved by using the compositions and methods of the present invention.

The combination of therapeutic agents may be used in the form of kits. The arrangement and construction of such kits is conventionally known to one of skill in the art. Such kits may include containers for containing the inventive combination of therapeutic agents and/or compositions, and/or other apparatus for administering the inventive combination of therapeutic agents and/or compositions.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

1. A method for treating a patient having a disease associated with over-expression of EZH2, comprising:
administering to the patient a therapeutically effective amount of a DNA methylation inhibitor.

2. The method of claim 1, further comprising:
detecting a level of EZH2 expression in the patient.

3. The method of claim 1, wherein the disease associated with over-expression of EZH2 is selected from the group consisting of: restenosis, benign tumor, cancer, hematologic disorder, and atherosclerosis.

4. The method of claim 3, wherein the cancer is an early stage cancer.

5. The method of claim 3, wherein the cancer is a late-stage cancer.

6. The method of claim 5, wherein the cancer is a late-stage, metastatic cancer.

7. The method of claim 3, wherein the benign tumor is selected from the group consisting of: hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

8. The method of claim 3, wherein the cancer is selected form the group consisting of: breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemia, lymphoma, B-Cell Non-Hodgkin's lymphoma, malignant melanoma, and epidermoid carcinoma.

9. The method of claim 3, wherein the hematological disorders are selected form the group consisting of acute myeloid leukemia, acute promyelocytic leukemia, acute lymphoblastic leukemia, chronic myelogenous leukemia, the myelodysplastic syndromes, and sickle cell anemia.

10. The method of claim 1, wherein the disease associated with over-expression of EZH2 is early-stage prostate cancer.

11. The method of claim 1, wherein the disease associated with over-expression of EZH2 is late-stage, metastatic prostate cancer.

12. The method of claim 1, wherein the disease associated with over-expression of EZH2 is early-stage non-Hodgkin's lymphoma.

13. The method of claim 1, wherein the disease associated with over-expression of EZH2 is late-stage non-Hodgkin's lymphoma.

14. The method of claim 2, wherein the detecting includes detecting the level of EZH2 expression in vivo in the patient.

15. The method of claim 2, wherein the detecting includes detecting the level of EZH2 expression ex vivo in the patient.

16. The method of claim 2, wherein the detecting includes detecting the level of EZH2 expression in a sample derived from the patient.

17. The method of claim 16, wherein the sample is derived from diseased tissue or organ of the patient.

18. The method of claim 16, wherein the sample is derived from the patient's prostate gland.

19. The method of claim 2, wherein the level of EZH2 expression is a level of EZH2 mRNA.

20. The method of claim 2, wherein the level of EZH2 expression is a level of EZH2 protein.

21. The method of claim 2, further comprising:
comparing the level of EZH2 expression in the patient with a level of EZH2 expression in a control sample.

22. The method of claim 21, wherein the control sample is obtained from a healthy tissue or organ of the patient.

23. The method of claim 21, wherein the control sample is obtained from a healthy individual.

24. The method of claim 2, wherein the detecting is performed prior to the administering of the DNA methylation inhibitor.

25. The method of claim 2, wherein the detecting is performed post the administering of the DNA methylation inhibitor.

26. The method of claim 2, wherein the detecting is performed both prior to and post the administering of the DNA methylation inhibitor.

27. The method of claim 2, wherein the therapeutically effective amount of the DNA methylation inhibitor is determined based upon the level of EZH2 expression in the patient.

28. The method of claim 1, wherein the DNA methylation inhibitor is a cytidine analog.

29. The method of claim 28, wherein the cytidine analog is 5-aza-cytidine.

30. The method of claim 28, wherein the cytidine analog is decitabine.

31. The method of claim 30, wherein decitabine is administered to the patient via an intravenous infusion at a dose of 2-50 mg/m² a day.

32. The method of claim 30, wherein decitabine is administered to the patient via an intravenous infusion at a dose of 5-20 mg/m² a day.

33. The method of claim 30, wherein decitabine is administered to the patient via an intravenous infusion at a dose of 1-100 mg/m² a day.

34. The method of claim 30, wherein decitabine is administered to the patient via an intravenous infusion at a dose of 1-100 mg/m² a day for at least 3 days per treatment cycle.

35. The method of claim 1, further comprising:
administering to the patient a therapeutically effective amount of a histone deacetylase inhibitor.

36. The method of claim 35, wherein the histone deacetylase inhibitor is trichostatin A.

37. The method of claim 36, wherein trichostatin A is administered to a patient by continuous intravenous infusion for at least 2-3 weeks at a dose of 100-2000 mg/m².

38. The method of claim 36, wherein trichostatin A is administered to a patient by continuous intravenous infulsion for at least 2-3 weeks at a dose of 250-1000 mg/m².

39. The method of claim 36, wherein trichostatin A is administered to a patient by continuous intravenous infusion for at least 2-3 weeks at a dose of 500-800 mg/m².

40. The method of claim 35, wherein the histone deacetylase inhibitor is depsipeptide and administered intravenously.

41. The method of claim 40, wherein depsipeptide administered to a patient by continuous intravenous infusion for at least 4 hours per day for a week at a dose of 2-100 mg/m².

42. The method of claim 40, wherein depsipeptide administered to a patient by continuous intravenous infusion for at least 4 hours per day for a week at a dose of 5-50 mg/m².

43. The method of claim 40, wherein depsipeptide administered to a patient by continuous intravenous infusion for at least 4 hours per day for a week at a dose of 5-15 mg/m².

44. The method of claim 35, wherein the histone deacetylase inhibitor is phenylbutyrate and administered intravenously.

45. The method of claim 44, wherein the phenylbutyrate is administered to a patient by continuous intravenous infusion for at least 2-3 weeks at a dose of 100-2000 mg/m².

46. The method of claim 44, wherein the phenylbutyrate is administered to a patient by continuous intravenous infusion for at least 2-3 weeks at a dose of 250-1000 mg/m².

47. The method of claim 44, wherein the phenylbutyrate is administered to a patient by continuous intravenous infusion for at least 2-3 weeks at a dose of 500-800 mg/m².

48. The method of claim 35, further comprising:
detecting a level of EZH2 expression in the patient prior to the administration of the DNA methylation inhibitor and the histone deacetylase inhibitor.

49. The method of claim 1, further comprising:
administering to the patient a therapeutically effective amount of an EZH2 antagonist.

50. The method of claim 49, wherein the EZH2 antagonist is an antisense nucleic acid, a ribozyme, a small interfering RNA, or a triple helix molecule against EZH2.

51. The method of claim 49, wherein the EZH2 antagonist is an antibody against EZH2.

52. The method of claim 49, wherein the EZH2 antagonist is administered prior to the DNA methylation inhibitor.

53. The method of claim 49, further comprising:
administering to the patient a therapeutically effective amount of a histone deacetylase inhibitor.

54. The method of claim 53, further comprising:
detecting a level of EZH2 expression in the patient prior to the administration of the DNA methylation inhibitor and the histone deacetylase inhibitor.

55. The method of claim 53, further comprising:
administering to the patient a therapeutically effective amount of an anti-neoplastic agent.

56. The method of claim 55, wherein the anti-neoplastic agent is selected from the group consisting of alkylating agents, antibiotic agents, retinoids, anti-metabolic agents, hormonal agents, plant-derived agents, anti-agiogenesis agents, and biologic agents.

57. The method of claim 55, wherein the anti-neoplastic agent is administered to the patient post the administration of the DNA methylation inhibitor.

58. The method of claim 55, further comprising:
detecting a level of EZH2 expression in the patient prior to the administration of the DNA methylation inhibitor and the histone deacetylase inhibitor.

59. A method for treating a patient having a disease associated with over-expression of EZH2, comprising:
administering to the patient a therapeutically effective amount of a histone deacetylase inhibitor.

60. The method of claim 59, further comprising:
detecting a level of EZH2 expression in the patient.

61. The method of claim 59, wherein the disease associated with over-expression of EZH2 is selected from the group consisting of: restenosis, benign tumors, cancer, hematologic disorders, and atherosclerosis.

62. The method of claim 61, wherein the cancer is an early-stage cancer.

63. The method of claim 61, wherein the cancer is a late-stage cancer.

64. The method of claim 63, wherein the cancer is a late-stage, metastatic cancer.

65. The method of claim 61, wherein the benign tumor is selected from the group consisting of: hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

66. The method of claim 61, wherein the cancer is selected form the group consisting of breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemia, lymphoma, B-Cell non-Hodgkin's lymphoma, malignant melanoma, and epidermoid carcinoma.

67. The method of claim 61, wherein the hematological disorders are selected form the group consisting of acute myeloid leukemia, acute promyelocytic leukemia, acute lymphoblastic leukemia, chronic myelogenous leukemia, the myelodysplastic syndromes, and sickle cell anemia.

68. The method of claim 59, wherein the disease associated with over-expression of EZH2 is early-stage prostate cancer.

69. The method of claim 59, wherein the disease associated with over-expression of EZH2 is late-stage metastatic prostate cancer.

70. The method of claim 59, wherein the disease associated with over-expression of EZH2 is early-stage non-Hodgkin's lymphoma.

71. The method of claim 59, wherein the disease associated with over-expression of EZH2 is late-stage non-Hodgkin's lymphoma.

72. The method of claim 60, wherein the detecting includes detecting the level of EZH2 expression in vivo in the patient.

73. The method of claim 60, wherein the detecting includes detecting the level of EZH2 expression ex vivo in the patient.

74. The method of claim 60, wherein the detecting includes detecting the level of EZH2 expression in a sample derived from the patient.

75. The method of claim 74, wherein the sample is derived from diseased tissue or organ from the patient.

76. The method of claim 74, wherein the sample is derived from the patient's prostate gland.

77. The method of claim 60, wherein the level of EZH2 expression is a level of EZH2 mRNA.

78. The method of claim 60, wherein the level of EZH2 expression is a level of EZH2 protein.

79. The method of claim 60, further comprising:
comparing the level of EZH2 expression in the patient with a level of EZH2 expression in a control sample.

80. The method of claim 79, wherein the control sample is located in a healthy tissue or organ of the patient.

81. The method of claim 79, wherein the control sample is obtained from a healthy individual.

82. The method of claim 60, wherein the detecting is performed prior to the administering of the histone deacetylase inhibitor.

83. The method of claim 60, wherein the detecting is performed post the administering of the histone deacetylase inhibitor.

84. The method of claim 60, wherein the detecting is performed both prior to and post the administering of the histone deacetylase inhibitor.

85. The method of claim 60, wherein the therapeutically effective amount of the histone deacetylase inhibitor is determined based upon the level of EZH2 expression in the patient.

86. The method of claim 59, wherein the histone deacetylase inhibitor is trichostatin A.

87. The method of claim 86, wherein trichostatin A is administered to a patient by continuous intravenous infusion for at least 2-3 weeks at a dose of 100-2000 mg/m².

88. The method of claim 86, wherein trichostatin A is administered to a patient by continuous intravenous infusion for at least 2-3 weeks at a dose of 250-1000 mg/m².

89. The method of claim 86, wherein trichostatin A is administered to a patient by continuous intravenous infusion for at least 2-3 weeks at a dose of 500-800 mg/m².

90. The method of claim 59, wherein the histone deacetylase inhibitor is depsipeptide and is administered intravenously.

91. The method of claim 90, wherein depsipeptide administered to a patient by continuous intravenous infusion for at least 4 hours per day for a week at a dose of 2-100 mg/m².

92. The method of claim 90, wherein depsipeptide administered to a patient by continuous intravenous infusion for at least 4 hours per day for a week at a dose of 5-50 mg/m².

93. The method of claim 90, wherein depsipeptide administered to a patient by continuous intravenous infusion for at least 4 hours per day for a week at a dose of 5-15 mg/m².

94. The method of claim 59, wherein the histone deacetylase inhibitor is phenylbutyrate and administered intravenously.

95. The method of claim 94, wherein the phenylbutyrate is administered to a patient by continuous intravenous infusion for at least 2-3 weeks at a dose of 100-2000 mg/m².

96. The method of claim 94, wherein the phenylbutyrate is administered to a patient by continuous intravenous infusion for at least 2-3 weeks at a dose of 250-1000 mg/m².

97. The method of claim 94, wherein the phenylbutyrate is administered to a patient by continuous intravenous infusion for at least 2-3 weeks at a dose of 500-800 mg/m².

98. The method of claim 59, further comprising:
administering to the patient a therapeutically effective amount of an EZH2 antagonist.

99. The method of claim 98, wherein the EZH2 antagonist is an antisense nucleic acid, a ribozyme, a small interfering RNA, or a triple helix molecule against EZH2.

100. The method of claim 98, wherein the EZH2 antagonist is an antibody against EZH2.

101. The method of claim 98, wherein the EZH2 antagonist is administered prior to the administering of the histone deacetylase inhibitor.

102. The method of claim 98, further comprising:
administering to the patient a therapeutically effective amount of an anti-neoplastic agent.

103. The method of claim 102, wherein the anti-neoplastic agent is selected from the group consisting of alkylating agents, antibiotic agents, retinoids, anti-metabolic agents, hormonal agents, plant-derived agents, anti-agiogenesis agents and biologic agents.

104. The method of claim 102, wherein the anti-neoplastic agent is administered to the patient post the administration of the histone deacetylase inhibitor.

105. The method of claim 102, wherein the EZH2 antagonist is administered to the patient prior to the administration of both the histone deacetylase inhibitor and the antineoplastic agent.
